SEQUENCE LISTING (3 sequences)

SEQ ID NO: 1 - protein, 426 aa, Variovorax paradoxus
SSGLVPRGSHMDSTLPVYSVAGAPEALALRAGPPASVRAALLAARRRTLDLADDFRAALGDAYPGIGYAPELNPPLWELGHVAWFQEWWIGRNRQRARGVACEPDHAREPSLLPQADAWYDSGRVAHRTRWALPLPDAEATRDYLERTLAQTLALLDELPPDAHDDALYFFRLVALHEAMHAEAAAYMAEGLGIALREGGVAPQLAEDAELELPAQRLRMGSDAGTGFAFDNELLSHDVSIEPLRIDAQAVSWARFLPFVEAGGYEHPAWWSDAGRDWLARQLLRHPAHLRAAGTGWQQRRGGRWLPLDPQGAAVHLNAHEAEAWCRWAGRRLPTEAEWECAALTLPGFAWGRVWEWTSSPFEPYPGFAPHPYRDYSAPWFGTRRVLRGACHATSAALAHARYRNFFEPHRRDIFAGFRSCRAPGG

SEQ ID NO: 2 - protein, 341 aa, Variovorax paradoxus
SSGLVPRGSHMSNPSLVIVSPALPGANNGNWRTAQRWKALLSPVCSARVVQQWPDADASADTVMLALHARRSAESIAHWAHAHPGRGLGVVLTGTDLYQDIGSDPQAQRSLQLAQRLVVLQALGAEALPPECRAKARVVYQSTSARAELPKSARQLRAVMVGHLRQVKSPQTLFDAARLLCGREDIRIDHIGDAGDAGLGELARALASDCPGYRWLGALPHAQTRQRIQRAHVLVHTSALEGGAHVIMEAVRSGTPVLASRVPGNVGMLGNDYAGYFPHGDAAALAALLEACRAGQGSKDRAAGLLDSLRTQCALRAPLFDPRAEQAALFQLLNELQPPPP

SEQ ID NO: 3 - protein, 367 aa, Variovorax paradoxus
SSGLVPRGSHMNAALPQAPAAEPRLTSLSHGGGCGCKIAPGVLSEILKGTARMPMPPELLVGIETADDAAVYQLNDEQALIATTDFFMPIVDDPFDFGRIAATNAISDVYAMGGKPILALALVGMPINVLSTATIGRILEGGESVCRAAGIPIAGGHTIDSVEAIYGLVALGLVHPKRVKRNAGAKAGDLLVLGKPLGVGILSAALKKEALDAAGYQRMIATTTRLNTSGPELAALDGVHALTDVTGFGLAGHALELARGAKLSLEIDWQQVPVLEGVRGLVQSGHVTGASGRNWAGYGSDVALPQNFAEEERALLTDPQTSGGLLVSCAPEALDEVLAIFRRHGFDDAAVIGTASAQGSAPRLVIR

(19) United States
(12) Patent Application Publication
(10) Pub. No.: US 2025/0327105 A1
(43) Pub. Date: Oct. 23, 2025

(54) SCALABLE, ECONOMICAL SYNTHESIS OF SELENONEINE AND ITS ANALOGS

(71) Applicant: THE TRUSTEES OF PRINCETON UNIVERSITY, Princeton,, NJ (US)

(72) Inventors: Mohammad R. SEYEDSAYAMDOST, Princeton, NJ (US); Chase M KAYROUZ, Plainsboro, NJ (US)

(73) Assignee: THE TRUSTEES OF PRINCETON UNIVERSITY, Princeton,, NJ (US)

(21) Appl. No.: 19/124,615

(22) PCT Filed: Oct. 26, 2023
(86) PCT No.: PCT/US2023/077824

§ 371 Date: Apr. 25, 2025

Related U.S. Application Data
(60) Provisional application No. 63/419,404, filed on Oct. 26, 2022.

(51) Int. Cl. C12P 17/10; C07K 14/195; C12N 1/20; C12N 9/02; C12N 15/70; C12R 1/19

(52) U.S. Cl. CPC C12P 17/10; C07K 14/195; C12N 1/20; C12N 9/0004; C12N 15/70; C12R 2001/19

(57) ABSTRACT

The present disclosure provides a rapid, facile, economical, and highly scalable method for producing selenoneine. The method involves SenA, hercynine, a selenosugar, and optionally a reductant, and a single, high-yielding enzymatic step in, e.g., a neutral-pH aqueous buffer and at ambient temperature. It can achieve yields of around 5 grams selenoneine per liter reaction with approximate costs of $100 per gram selenoneine.

### CROSS-REFERENCE TO RELATED APPLICATIONS

The present application claims priority to U.S. Provisional Patent Application No. 63/419,404, filed Oct. 26, 2022, the contents of which are incorporated by reference herein in its entirety.

### STATEMENT REGARDING FEDERALLY SPONSORED RESEARCH OR DEVELOPMENT

This invention was made with government support under Grant No. GM140034 awarded by the National Institutes of Health. The government has certain rights in the invention.

### SEQUENCE LISTING

The present application contains a Sequence Listing which has been submitted electronically in ST.26 Sequence listing XML format and is hereby incorporated by reference in its entirety. Said ST.26 Sequence listing XML, created on Oct. 11, 2023, is named PRIN-91876_ST26.xml and is 4,654 bytes in size.

### TECHNICAL FIELD

The present application is drawn to techniques for synthesizing selenoneine and its analogs, and chemoenzymatic synthesizing of those compounds in particular.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Selenoneine and its analogs are important antioxidants with vitamin-like and therapeutic properties. When compared to its well-commercialized sulfur analog, ergothioneine, selenoneine exhibits an enhanced radical scavenging activity, methylmercury detoxification functionality, and resistance to oxidative degradation. The myriad cytoprotective properties of selenoneine have been known for some time. However, how cells synthesize this molecule has remained elusive. Methods have been developed for the chemical production of selenoneine. However, these utilize harsh chemicals as the reactive element selenium must be incorporated in a specific fashion.

### BRIEF SUMMARY

Various deficiencies in the prior art are addressed below by the disclosed compositions of matter and techniques.

In various aspects, a process for producing selenoneine or an analog thereof may be provided. The process may include providing a recombinant microorganism (such as an E. coli strain) configured to express a SenA protein [SEQ ID NO: 1] or analog thereof. The process may include preparing a cell-free lysate of the modified microorganism. The cell-free lysate may include the SenA protein in a fluid (such as a neutral-pH aqueous buffer). The SenA protein may be soluble in the fluid. The process may include enzymatically generating selenoneine by adding additional materials to the cell-free lysate. The additional materials may include hercynine and a selenosugar (such as 1-seleno-N-acetyl-β-D-glucosamine). The additional materials may include a reductant (such as dithiothreitol). In some embodiments, enzymatically generating selenoneine may be performed at a temperature of 20-25° C.

The process may include incubating the modified microorganism. Providing the modified microorganism may include providing a base microorganism strain, and then introducing DNA which encodes a polypeptide sequence configured to express the SenA protein into the base microorganism strain.

The process may include purifying the selenoneine (e.g., via chromatography). The process may include chemically derivatizing seleno-containing substrates and products with a reactant (such as monobromobimane).

In various aspects, an engineered microorganism (such as a strain of E. coli) may be provided. The microorganism may include a DNA sequence configured to express a SenA protein [SEQ ID NO: 1] or an analog thereof. The microorganism may be free of sequences configured to express a SenB protein [SEQ ID NO: 2] or an analog thereof and sequences configured to express a SenC protein [SEQ ID NO: 3] or an analog thereof.

In various aspects, an intermediate composition may be provided. The intermediate composition may include a cell-free lysate including a SenA protein [SEQ ID NO: 1] or an analog thereof in a fluid. The SenA protein may be soluble in the fluid. The cell-free lysate may be free of SenB protein [SEQ ID NO: 2] or analogs thereof and SenC protein [SEQ ID NO: 3] or analogs thereof.

In various aspects, an intermediate composition may be provided. The intermediate composition may include hercynine and a selenosugar. The intermediate composition may include a reductant. A ratio of concentrations of hercynine to selenosugar to reductant may be about 2:2:1 in the intermediate composition.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart of a method.

FIG. 2 is a graph showing the result of a typical reaction containing 20 mM hercynine, 20 mM 1-seleno-N-acetyl-β-D-glucosamine (monomer basis), and 10 mM dithiothreitol in 0.1 mL of cell-free E. coli lysate containing recombinant SenA.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Selenoneine and its analogs are important antioxidants with vitamin-like and therapeutic properties. When compared to its well-commercialized sulfur analog, ergothioneine, selenoneine exhibits an enhanced radical scavenging activity, methylmercury detoxification functionality, and resistance to oxidative degradation. Disclosed herein is scalable, economical synthesis of selenoneine and its analogs.

As used herein, the term “analog” refers to a molecule which is structurally similar or shares similar or corresponding attributes with another molecule. As used herein, the term “analog [of a protein]” refers to any polypeptide that is structurally similar to the protein in question, and that shares the biochemical or biological activity of the protein in question upon which the analog is based. Various references disclose modification of polypeptides by polymer conjugation or glycosylation. The term analog includes polypeptides conjugated to a polymer such as PEG and may be comprised of one or more additional derivatizations of cysteine, lysine, or other residues. In addition, analogs of the instant invention may comprise a linker or polymer, wherein the amino acid to which the linker or polymer is conjugated may be a non-natural amino acid, or may be conjugated to a naturally encoded amino acid utilizing techniques known in the art such as coupling to lysine or cysteine. encompasses the conventional and well-known naturally occurring amino acids, as well as all synthetic variations and derivatives thereof. In some embodiments, the analogs comprise alanine, arginine, asparagine, aspartic acid, cysteine, glutamine, glutamic acid, glycine, bistidine, isoleucine, leucine, lysine, methionine, phenylalanine, proline, serine, threonine, tryptophan, tyrosine, and/or valine substitutions. In some embodiments, analogs comprise N-methylated α-amino acids, hydroxylated amino acids, and aminooxy acids. In some embodiments, analogs comprise N-alkyl amino acids (such as N-methyl glycine), hydroxylysine, 3-hydroxyproline, 4-hydroxyproline, nor-valine, nor-leucine, and orithine. The terms “amino acid” refer to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs including non-natural analogs.

The myriad cytoprotective properties of selenoneine have been known for some time. Methods have been developed for the chemical production of selenoneine. However, these utilize harsh chemicals as the reactive element selenium has to be incorporated in a specific fashion. Moreover, these methods are very low-yielding and not suitable for scalable production of selenoneine.

The genes required for selenoneine production in diverse microbes was recently identified and an enzymatic synthesis of this important molecule was recently disclosed (see PCT/US2023/016183, incorporated by reference herein in its entirety), wherein three different enzymes are utilized to generate selenoneine. The present application improves upon that approach by simplifying the process further to make it scalable, economical, and rapid.

The process disclosed herein can be used to generate large amounts of selenoneine and its analogs. The process is ‘green’ in that it uses an enzyme for key transformations rather than harsh chemicals. With this approach, variants of selenoneine may be generated and tested in diverse assays.

The method disclosed herein uses only one enzyme, SenA, in an unpurified crude extract from a microorganism. Thus, in various aspects, a process for producing selenoneine or an analog thereof may be provided.

The structure of selenoneine is shown below.

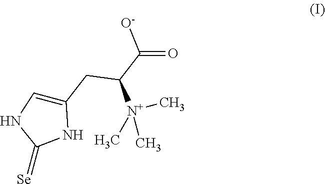

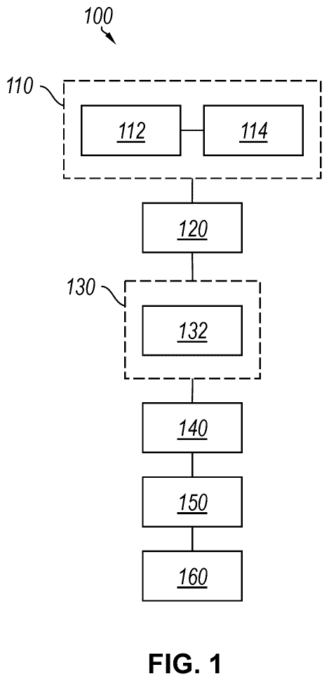

Referring to FIG. 1, the process 100 may include providing 110 a modified microorganism (e.g., a recombinant microorganism) configured to express a SenA protein [SEQ ID NO: 1] or analog thereof. In some embodiments, the microorganisms may have already been prepared. In some embodiments, the microorganisms may be provided by first providing 112 a base microorganism strain, and then introducing 114 DNA which encodes a polypeptide sequence configured to express the SenA protein into the base microorganism strain. Such techniques are well understood in the art.

While E. coli was in various experiments disclosed herein, it will be understood that various other microorganisms may be readily utilized to express SenA. Useful microorganisms include algae, yeast and bacteria. The recombinant microorganism may be a gram-negative bacterium, e.g., E. coli, Salmonella, Rhodobacter, Synechtocystis, or Erwinia. Other gram-negative bacteria include members from the Methylococcaceae and Methylocystaceae families; Thermotoga hypogea, Thermotoga naphthophila, Thermotoga subterranean, Petrotoga halophila, Petrotoga mexicana, Petrotoga miotherma, and Petrotoga mobilis. The recombinant microorganism may be a gram-positive bacterium, e.g., B. subtilis or Clostridium. The recombinant microorganism may be a fungus such as Saccharomyces cerevisae, Pichia pastoris, Hansenula polymorpha, Kluyveromyces lactis, Yarrowia lipolytica, Scizosacchromyces pombe or Trichoderma reesei or other yeast species of genus Saccharomyces, Pichia, Hansenula, Kluyveromyces, Yarrowia, Trichoderma or Scizosacchromyces. The recombinant microorganism may also be a eukaryote such as an algae. Example of algae include Chlamydomonas.

The SenA protein or analog thereof is preferably a SenA protein [SEQ ID NO: 1], 426 amino acids in length. However, in some embodiments, a protein with at least a 99% sequence identity to the SenA protein [SEQ ID NO: 1] may be utilized. In some embodiments, a single addition, substitution, or deletion mutation may exist in the analog. In some embodiments, two or fewer addition, substitution, or deletion mutations may exist in the analog. In some embodiments, three or fewer addition, substitution, or deletion mutations may exist in the analog. In some embodiments, four or fewer addition, substitution, or deletion mutations may exist in the analog. In some embodiments, there are no mutations in positions 1-50. In some embodiments, there are no mutations in positions 51-100. In some embodiments, there are no mutations in positions 101-200. In some embodiments, there are no mutations in positions 201-300. In some embodiments, there are no mutations in positions 301-350. In some embodiments, there are no mutations in positions 351-426.

The SenA protein or analog thereof may be fused to one or more tags.

The tag(s) may include a purification tag. As used herein, the term “purification tag” preferably refers to an additional amino acid sequence (a peptide or polypeptide) which allows for purification of the SenA protein or analog thereof. Non-limiting examples of purification tags include polyhistidine tag, polyarginine tag, glutathione-S-transferase (GST), maltose binding protein (MBP), influenza virus HA tag, thioredoxin, staphylococcal protein A tag, the FLAG™ epitope, and the c-myc epitope. In a preferred embodiment, the purification tag is a polyhistidine tag. In a more preferred embodiment, the polyhistidine tag comprises at least 6 consecutive histidine residues.

The tag(s) may include a fluorescent protein or tag. Non-limiting examples of fluorescent proteins include green fluorescent proteins (e.g., GFP, eGFP, GFP-2, tagGFP, turboGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., BFP, EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HoRed-Tandem, HcRod1, AsRed2, eqFP611, mRasberry, mStrawberry, Jred), and orange fluorescent proteins (e.g., mOrange, mKO, Kusabira-Orange, Monomeric Kusabira-Orange, m Tangerine, tdTomato).

The process may include incubating 120 the modified microorganism, during which time the SenA may be expressed by the microorganism. Such techniques are well understood, and appropriate growth media and incubation conditions are well known.

The process may include preparing 130 a cell-free lysate of the modified microorganism. Such techniques are well known in the art. The process may include lysing 132 the microorganism. Lysing techniques are well known in the art, and may include, e.g., sonication, homogenization, freeze-thaw lysis, high-temperature lysis, enzymatic lysis, and/or chemical lysis. This may include centrifugation.

For example, 6×His-tagged SenA was produced in E. coli BL21 (DE3) cells grown in two 4 L flasks, each containing 2 L Terrific Broth supplemented with 50 mg/L kanamycin at 37° C./170 rpm. Small cultures were prepared by inoculating 40 mL of LB medium containing 50 mg/L Kan with a single colony of E. coli BL21 (DE3) carrying a desired plasmid. After overnight growth at 37° C./170 rpm, 4 L of TB medium plus 50 mg/L Kan were inoculated with the 40 mL small culture and incubated at 37° C./170 rpm. At OD₆₀₀=0.5-0.6, protein expression was induced with 0.2 mM IPTG, and cultures were incubated at 37° C./170 rpm for an additional 12-24 hours. Cells were pelleted by centrifugation (8,000 g, 15 min, 4° C.), yielding ˜7 g of cell paste per L. The cell pastes were stored at −80° C. until purification.

All purification steps were carried out in a cold room at 4° C. Cells were resuspended in lysis buffer (5 mL/g cell paste), which consisted of 25 mM Tris-HCl, 300 mM NaCl, 10 mM imidazole, 10% glycerol, pH=7.7, supplemented with 1 μL/mL Protease Inhibitor Cocktail (from Sigma) and 1 mM phenylmethylsulfonyl fluoride. Once homogenous, 0.1 mg/mL deoxyribonuclease I (from Alfa Aesar) was added, and the cells were lysed by the addition of 5 mg/mL lysozyme followed by sonication using 30% power (˜150 W) in 15 s on/15 s off cycles for a total of 4 min. This process was repeated twice. The lysate was then clarified by centrifugation (17,000 g, 15 min, 4° C.) and loaded onto a 5 mL Ni-NTA column pre-equilibrated in lysis buffer. The column was washed with lysis buffer and His-tagged proteins were eluted with elution buffer consisting of 25 mM Tris-HCl, 300 mM NaCl, 300 mM imidazole, 10% glycerol, pH=7.7. Eluted proteins were then buffer-exchanged using a 50 mL column of SEPHADEX® G-25 medium (from Cytiva) into storage buffer consisting of 25 mM Tris-HCl, 150 mM NaCl, 10% glycerol, pH=7.7. Purified proteins were stored at −80° C. Protein concentrations were determined spectrophotometrically on a CARY® 60 UV-visible spectrophotometer (from Agilent) using calculated molar extinction coefficients at 280 nm. From 4 L cultures, 105 mg SenA yields were obtained.

The cell-free lysate may include the SenA protein in a fluid. The SenA is preferably soluble in the fluid. The fluid may be a buffer, such as an aqueous buffer, such as a neutral-pH aqueous buffer.

SenA is a remarkable enzyme that can be used to join N,N,N-trimethyl-L-histidine (hercynine) with a selenosugar to generate selenoneine or an analog thereof. When SenA, hercynine, and 1-seleno-N-acetyl-β-D-glucosamine (the selenosugar) are utilized, selenoneine may be formed. Other selenosugars may generate analogs of selenoneine.

Thus, the process may include enzymatically generating 140 selenoneine or an analog thereof by adding additional materials to the cell-free lysate. The additional materials may include hercynine and a selenosugar.

The selenosugar may be acetylated or non-acetylated 1-selenosugar. The sugar may be a monosaccharide or a polysaccharide. The sugar may be an aminosugar. Non-limiting examples of non-acetylated selenosugars include 1-selenoglucose, 1-selenomannose, 1-selenogalactose, 1-selenoribose, 1-selenomaltose and 1-selenofucose. Non-limiting examples of acetylated selenosugars include 1-seleno-N-acetyl-β-D-glucosamine, 1β-Methylseleno-N-acetyl-D-galactosamine.

The additional materials may include a reductant. As used herein, the term “reductant” refers to any material that is capable of either directly (chemically) or indirectly (via biological systems) of donating electrons to impact a reduction reaction as part of the reaction to generate selenoneine or an analog thereof. It will be understood that the reductant may be inorganic or organic. Non-limiting examples of a reductant include dithiothreitol (DTT), mercaptoethanol, cysteine, thioglycolate, cysteamine, glutathione, and sodium borohydride.

In some embodiments, enzymatically generating selenoneine may be performed at a temperature above 25° C. In some preferred embodiments, enzymatically generating selenoneine may be performed at a temperature of 20-25° C.

The process may include chemically derivatizing 150 selenol-containing substrates and products with a reactant (such as monobromobimane).

The process may include purifying 160 the selenoneine (e.g., via chromatography). Such techniques are well known in the art.

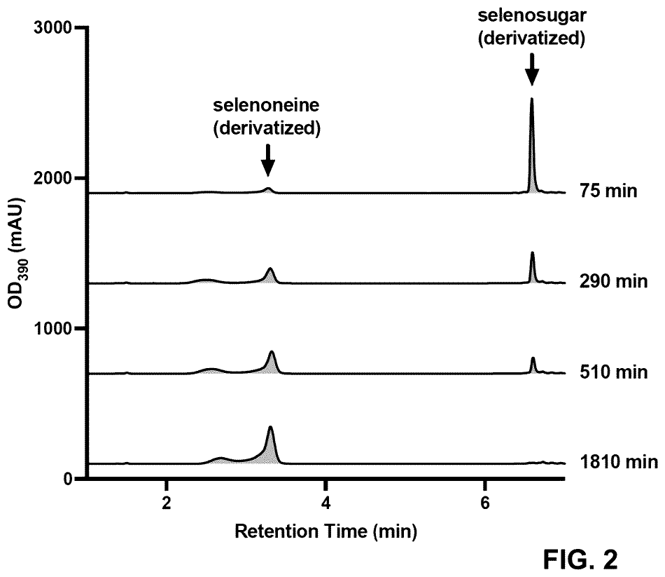

Referring to FIG. 2, depicted is the result of a typical reaction containing 20 mM hercynine, 20 mM 1-seleno-N-acetyl-β-D-glucosamine (monomer basis), and 10 mM dithiothreitol in 0.1 mL of cell-free E. coli lysate containing recombinant SenA. Reaction progress was monitored by chemical derivatization of selenol-containing substrates and products with monobromobimane, followed by HPLC-UV analysis. As shown, near-quantitative conversion is achieved between 510 and 1810 minutes of reaction time.

In various aspects, an engineered microorganism (such as a strain of E. coli) may be provided. As disclosed herein the microorganism may include an algae, yeast, or bacteria. The recombinant microorganism may be a gram-negative bacterium, e.g., E. coli, Salmonella, Rhodobacter, Synechtocystis, or Erwinia. Other gram-negative bacteria include members from the Methylococcaceae and Methylocystaceae families; Thermotoga hypogea. Thermotoga naphthophila, Thermotoga subterranean, Petrotoga halophila, Petrotoga mexicana, Petrotoga miotherma, and Petrotoga mobilis. The recombinant microorganism may be a gram-positive bacterium, e.g., B. subtilis or Clostridium. The recombinant microorganism may be a fungus such as Saccharomyces cerevisae, Pichia pastoris, Hansenila polymorpha, Kluyveromyces lactis, Yarrowia lipolytica, Scizosacchromyces pombe or Trichoderma reesei or other yeast species of genus Saccharomyces, Pichia, Hansemda, Kluyveromyces, Yarrowia, Trichoderma or Scizosacchromyces. The recombinant microorganism may also be a eukaryote such as an algae. Example of algae include Chlamydomonas.

The microorganism may include a DNA sequence configured to express a recombinant SenA protein [SEQ ID NO: 1] or an analog thereof.

As disclosed herein, the SenA protein or analog thereof is preferably a SenA protein [SEQ ID NO: 1], 426 amino acids in length. However, in some embodiments, an analog of SenA may have at least a 99% sequence identity to the SenA protein [SEQ ID NO: 1] may be utilized. In some embodiments, a single addition, substitution, or deletion mutation may exist in the analog. In some embodiments, two or fewer addition, substitution, or deletion mutations may exist in the analog. In some embodiments, three or fewer addition, substitution, or deletion mutations may exist in the analog. In some embodiments, four or fewer addition, substitution, or deletion mutations may exist in the analog. In some embodiments, there are no mutations in positions 1-50. In some embodiments, there are no mutations in positions 51-100. In some embodiments, there are no mutations in positions 101-200. In some embodiments, there are no mutations in positions 201-300. In some embodiments, there are no mutations in positions 301-350. In some embodiments, there are no mutations in positions 351-426.

The SenA or analog thereof may be fused to one or more tags, as disclosed herein.

The microorganism may be free of sequences that express molecules that are configured to interfere with SenA's interaction with hercynine and a selenosugar. For example, the microorganism may be free of genes encoding sequences involved in enzymatic synthesis of selenoneine. More specifically, the microorganism may be free of sequences configured to express a recombinant SenB protein [SEQ ID NO: 2] or an analog thereof and sequences configured to express a recombinant SenC protein [SEQ ID NO: 3] or an analog thereof. In some embodiments, the microorganism may be free of sequences configured to express a protein having at least 99% sequence identity to SenB [SEQ ID NO: 2] and sequences configured to express a protein having at least 99% sequence identity to SenC [SEQ ID NO: 3].

In various aspects, an intermediate composition may be provided. The intermediate composition may include a cell-free lysate including a recombinant SenA protein [SEQ ID NO: 1] or an analog thereof in a fluid.

As disclosed herein, the SenA protein or analog thereof is preferably a SenA protein [SEQ ID NO: 1], 426 amino acids in length. However, in some embodiments, an analog of SenA may have at least a 99% sequence identity to the SenA protein [SEQ ID NO: 1] may be utilized. In some embodiments, a single addition, substitution, or deletion mutation may exist in the analog. In some embodiments, two or fewer addition, substitution, or deletion mutations may exist in the analog. In some embodiments, three or fewer addition, substitution, or deletion mutations may exist in the analog. In some embodiments, four or fewer addition, substitution, or deletion mutations may exist in the analog. In some embodiments, there are no mutations in positions 1-50. In some embodiments, there are no mutations in positions 51-100. In some embodiments, there are no mutations in positions 101-200. In some embodiments, there are no mutations in positions 201-300. In some embodiments, there are no mutations in positions 301-350. In some embodiments, there are no mutations in positions 351-426.

The SenA or analog thereof may be fused to one or more tags, as disclosed herein.

The fluid may be a buffer, such as an aqueous buffer, such as a neutral-pH aqueous buffer.

The recombinant SenA protein or analog thereof may be soluble in the fluid. The cell-free lysate may be free of recombinant SenB protein [SEQ ID NO: 2] or analogs thereof and recombinant SenC protein [SEQ ID NO: 3] or analogs thereof. The cell-free lysate may be free of proteins having at least 99% sequence identify to SenB [SEQ ID NO: 2] and proteins having at least 99% sequence identify to SenC [SEQ ID NO: 3].

In various aspects, an intermediate composition may be provided. The intermediate composition may include hercynine and a selenosugar. The selenosugar may be any selenosugar as disclosed herein. The intermediate composition may include a reductant as disclosed herein.

In various embodiments, a ratio of concentrations of hercynine to selenosugar to reductant may be about 2:2:1 in the intermediate composition. The term “about” is used to indicate that each value in the ratio may vary by ±10% (e.g., the ratio could be 2.2:1.8:1). In some embodiments, a ratio of hercynine to reductant may be at least 1.5:1. In some embodiments, a ratio of hercynine to reductant may be at least 2:1. In some embodiments, a ratio of hercynine to reductant may be 5:1-1.5:1. In some embodiments, a ratio of selenosugar to reductant may be at least 1.5:1. In some embodiments, a ratio of selenosugar to reductant may be at least 2:1. In some embodiments, a ratio of selenosugar to reductant may be 5:1-1.5:1. In some embodiments, a ratio of hercynine to selenosugar may be 1.5:1-1:1.5.

In various examples, the disclosed simple chemical synthesis of hercynine and the selenosugar is combined with even crude SenA to deliver selenoneine in excellent yields (e.g., at least 5 g material per liter).

This is a very cost-effective technique; it is estimated that the cost of selenoneine generated in this fashion should be $100 per gram material. As comparison, Sigma-Aldrich sells ergothioneine (the simpler sulfur variant of selenoneine) at $1,110 per 25 milligrams.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A process for producing selenoneine, comprising:
providing a recombinant microorganism configured to express a SenA protein;
preparing a cell-free lysate of the recombinant microorganism, the cell-free lysate including the SenA protein in a fluid, the SenA protein being soluble in the fluid; and
enzymatically generating selenoneine by adding additional materials to the cell-free lysate, the additional materials including hercynine and a selenosugar.

2. The process of claim 1, wherein the recombinant microorganism is E. coli.

3. The process of claim 1, wherein the selenosugar is 1-seleno-N-acetyl-β-D-glucosamine.

4. The process of claim 1, wherein the additional materials added to the cell-free lysate further includes a reductant.

5. The process of claim 4, wherein the reductant is dithiothreitol.

6. The process of claim 1, further comprising incubating the recombinant microorganism.

7. The process of claim 1, wherein providing a recombinant microorganism expressing a SenA protein includes:
providing a base microorganism strain; and
introducing DNA which encodes a polypeptide sequence configured to express the SenA protein into the base microorganism strain.

8. The process of claim 1, further comprising purifying the selenoneine.

9. The process of claim 8, wherein the selenoneine is purified via chromatography.

10. The process of claim 1, wherein the fluid is a neutral-pH aqueous buffer.

11. The process according to claim 1, wherein enzymatically generating selenoneine is performed at a temperature of 20-25° C.

12. The process according to claim 1, further comprising chemically derivatizing selenolcontaining substrates and products with a reactant.

13. The process according to claim 12, wherein the reactant is monobromobimane.

14. An engineered microorganism, comprising:
a DNA sequence configured to express a SenA protein, wherein the engineered microorganism is free of sequences configured to express a SenB protein and a SenC protein.

15. The engineered microorganism of claim 14, wherein the engineered microorganism is a strain of E. coli.

16. An intermediate composition, comprising:
cell-free lysate including a SenA protein in a fluid, the SenA protein being soluble in the fluid, the cell-free lysate being free of a SenB protein and a SenC protein.

17. An intermediate composition, comprising hercynine and a selenosugar.

18. The intermediate composition of claim 17, further comprising a reductant.

19. The intermediate composition of claim 18, wherein a ratio of concentrations of hercynine to selenosugar to reductant is about 2:2:1 in the intermediate composition.
